(19) United States
(12) Patent Application Publication
(10) Pub. No.: US 2025/0032699 A1
(43) Pub. Date: Jan. 30, 2025

(54) MEDICAL DEVICE FOR PREVENTING SURGICAL INFECTION

(71) Applicant: Nicholas Ian Duff, Plymouth, MI (US)

(72) Inventor: Nicholas Ian Duff, Plymouth, MI (US)

(21) Appl. No.: 18/787,671

(22) Filed: Jul. 29, 2024

Related U.S. Application Data
(60) Provisional application No. 63/529,227, filed on Jul. 27, 2023.

(51) Int. Cl. A61M 5/00; A61J 1/20; A61K 31/7036; A61K 38/14

(52) U.S. Cl. CPC A61M 5/002; A61J 1/2048; A61J 1/2096; A61K 31/7036; A61K 38/14

(57) ABSTRACT

A formatting system for a medical device for preventing surgical infection is disclosed, including a kit comprising a formatting system. The formatting system includes a first syringe having a sufficient amount of calcium sulfate and a second syringe including a sufficient amount of water, the kit including a case to provide a protective and sterile housing. A sterile tray is molded to an interior of the case to provide a mixing vessel to mix at least one medication with the sufficient amount of water via a mixing device. The second syringe and the first syringe are connectable to permit the mixing the of the at least one medication, the sufficient amount of water, and the calcium sulfate to form a paste. An elongated tip facilitates the application of the paste to a patient.

### CROSS-REFERENCE TO RELATED APPLICATIONS

The present application claims priority to U.S. Provisional Application No. 63/529,227 filed Jul. 27, 2023, titled “DRUG-LOADED MEDICAL DEVICE FOR PREVENTING SURGICAL INFECTION,” which is hereby incorporated by reference in its entirety.

### TECHNICAL FIELD

The embodiments disclosed herein generally relate formatting systems for a medical device for preventing surgical infection.

### BACKGROUND

Infections from various agents are common throughout life. The risk of infection is much greater during surgical procedures when the body's natural protective barrier against infection (e.g., the skin) is breached. To prevent infection, proper sanitization of the surgical area is necessary.

Many antibiotics have been identified and developed in recent years. The term antibiotic relates to antimicrobial substances which are active against bacteria and other microbes to kill or inhibit the growth of the infectious agent. Their development and use has had a significant impact on global health. In particular, Vancomycin is an antibiotic medication used to treat infections caused by Gram-positive bacteria. It is provided often provided as a liquid and is commonly administered through the intravenous and oral administration routes. Gentamicin is another common antibiotic used to treat a wide range of infections caused by (mostly) Gram-negative bacteria. It is effective when administered through topical, intravenous injection and intramuscular injection routes.

Calcium sulfate) is a common ingredient used in medicine and has various medicinal uses. In particular, calcium sulfate is known for its use in the preparation and delivery of antibiotic-loaded calcium sulfate beads which used as a treatment method for local antibiotic delivery.

Current devices do not include antibiotics. The antibiotics are pulled from the hospital's pharmacy which creates an extra step for the circulating nurse and for the surgeon to order the drugs. This also consumes critical resources including pharmacy. This takes critical time and concentration away from the patient. It is known that post-operative surgical infections may cause harm to the patient in addition to increased cost to the healthcare system. Known methods and devices in the current arts do not adequately reduce post-operative infections and need improvement.

The process of making antibiotic beads is cumbersome. Usually, there is a wait in initially getting the antibiotics. This requires almost 10-15 minutes to set up the product. The biodegradable material is mixed in a small bowl which does not keep all the product contained and there is usually spillage. Once the product is mixed into a paste on the small bowl it has to be spread on a rubber mold containing the bead holes. This takes at least 6-7 minutes and another 10 minutes for the product to cure and set. In highly complex cases the surgeon does not want the surgical technician to be distracted for considerable time on the back table. Also, the correct amount of drugs used are not concise during the mixing stage as many surgical technicians are estimating the dosage for smaller sized kits for which there is no clearly defined set-up protocol.

### SUMMARY OF THE INVENTION

This summary is provided to introduce a variety of concepts in a simplified form that is further disclosed in the detailed description of the embodiments. This summary is not intended for determining the scope of the claimed subject matter.

In general, the embodiments provided herein relate to a formatting system for a medical device for preventing surgical infection is disclosed, including a kit comprising a formatting system. The formatting system includes a first syringe having a sufficient amount of calcium sulfate and a second syringe including a sufficient amount of water, the kit including a case to provide a protective and sterile housing. A sterile tray is molded to an interior of the case to provide a mixing vessel to mix at least one medication with the sufficient amount of water via a mixing device. The second syringe and the first syringe are connectable to permit the mixing the of the at least one medication, the sufficient amount of water, and the calcium sulfate to form a paste. An elongated tip facilitates the application of the paste to a patient.

The formatting system allows for the selection of medications by a pharmacist or medical professional prior to the formatting system's delivery to the operating room. In such, the medical professionals can select medications which are most suitable for the particular application. This may be decided based on various factors including drug interactions, use case, patient allergies, etc.

In one particular use case, the formatting system may be utilized as a bone void filler which may prevent surgical infections caused as a result of infection at the surgical site during a medical procedure.

In one aspect, the formatting system includes a first syringe including a sufficient amount of vancomycin powder and calcium sulfate. A second syringe includes a sufficient amount of gentamicin, wherein the first syringe and the second syringe are connected via a connection luer lock device.

The operation of the device is to treat and prevent infection. In some aspects, the device may be pre-loaded with one or more medications. The biodegradable material can be executed in a flowable, workable fashion. The device may be set up and prepared about fifteen times faster than the current industry standard. Hospital systems will benefit substantially due to their decline in surgically related infections.

In one aspect, the effective amount of Vancomycin powder for a 5 cc kit is about 0.5 mg.

In one aspect, the effective amount of calcium sulfate for the 5 cc kit is about 15 ml.

In one aspect, the effective amount of Gentamicin for the 5 cc kit is about 3 ml.

In one aspect, the effective amount of water is about 12 ml.

In one aspect, the connection device is constructed as a luer locking mechanism.

In one aspect, the connection device includes a first end to lockingly attach to the first tip of the first syringe, and a second end to lockingly attach to the second tip of the second syringe, wherein the connection device provides a fluid connection between the first syringe and the second syringe.

In one aspect, a first plunger is connected to the first syringe and a second plunger is connected to the second syringe. The plungers are operable to forcibly mix the effective amount of Vancomycin powder, the effective amount of calcium sulfate, the effective amount of water, and the effective amount of Gentamicin.

In one aspect, the system includes an elongated tip which is effective for applying a bead formed by the paste to a specific anatomical location on the patient.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1A illustrates a front elevation view of a first syringe of the formatting system for a drug-loaded medical device for preventing surgical infection, wherein the first syringe is loaded with Vancomycin powder and calcium sulfate, according to some embodiments;

FIG. 1B illustrates a front elevation view of a second syringe of the formatting system for a drug-loaded medical device for preventing surgical infection, wherein the second syringe is loaded with Gentamicin, according to some embodiments;

FIG. 2 illustrates a perspective view of the connection device, according to some embodiments;

FIG. 3 illustrates a perspective view of the elongated tip, according to some embodiments;

FIG. 4 illustrates a perspective view of the first syringe and the second syringe connected via the connection device to allow the active ingredients to be mixed as is described by the formatting system for a drug-loaded medical device, according to some embodiments;

FIG. 5 illustrates a flowchart of a process for preparing and applying the formatting system for a drug-loaded medical device, according to some embodiments;

FIG. 6 illustrates a perspective view of the kit including the first syringe, the second syringe, the mixing device, and the sterile tray, according to some embodiments;

FIG. 7 illustrates the first syringe and the second syringe in an interlocked configuration, according to some embodiments; and

FIG. 8 illustrates a method of utilizing the kit, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relates to a formatting device for a medical device for the treatment and preventions of bacteria-related infections. The objective of the device and technique is to treat and prevent infection in the surgical setting. The system is provided such that the medical personnel can selectively load medications prior to the device being utilized in the operating room. The system is provided as a kit having two syringes which can each be loaded with one or more medications selected by a pharmacist or other medical personnel. In such, the system provides a customizable kit which can be pre-loaded with medications prior to its use in the operating room. The ability for the medical personnel or pharmacist to select the medications which are loaded into the system allows for a wide range of use-cases which may be dependent on the patients known drug interactions, application site where the medications will be applied, infectious agent, etc.

In some embodiments, the device may be utilized as a bone void filler. However, one skilled in the arts will readily understand that the device may have various additional use cases and it is to be understood that any mention of using the device as a bone void filler is utilized by way of a non-limiting example.

In some embodiments, the kit includes a first syringe, a second syringe, a mixing device, and a sterile tray. The kit components are provided in a case which provides a sterile and protective housing to retain the components.

In some embodiments, a tube of sterile water is provided with the kit. The sterile water is provided such that it can be mixed with the medications within the sterile tray. In one particular example, the tube contains about 24 ml of sterile water. However, one skilled in the arts will readily understand that various amounts of sterile water may be included in the kit. The mixing device may be used to mix the sterile water with the medications within the sterile tray.

In some embodiments, the first syringe may be filled with 30 ml of calcium sulfate, while the second syringe is empty at the time of manufacture and packaging of the kit. The empty second syringe is used to draw one or more medications into its interior, as well as to draw the sterile water into its interior.

In some embodiments, the first syringe and second syringe are configured to interlock with one another to allow for mixing of the medications contained within the first syringe and the second syringe.

In some embodiments, the device is comprised of an antibiotic impregnated calcium sulfate. In one example, the antibiotics (i.e., the composition) may include Vancomycin and Gentamicin. The antibiotic composition is mixed and delivered through a percutaneous custom syringe. An elongated tip allows for a flowable and workable paste giving surgeons the ability to utilize a precise release of the drug. This paste provides a means for pinpointing the location of the infection site more precisely than previous methods.

By mixing the product in two contained syringes the biodegradable material is mixed in significantly faster (e.g., about fifteen times faster in one example). This process also allows less spillage, and a more exact concentration of antibiotics is provided resulting in a safer and more efficient outcome for the patient. The system increases efficiencies in the operating room by streamlining set up and the ability to access challenging areas of the anatomy.

As used herein, the term “active ingredients” are used to describe the compounds which include antibiotic or otherwise pharmaceutically active properties. This may include the Gentamicin, Vancomycin, and calcium sulfate components of the formatting system. Water is also a component of the formatting system and may be included with the active ingredients.

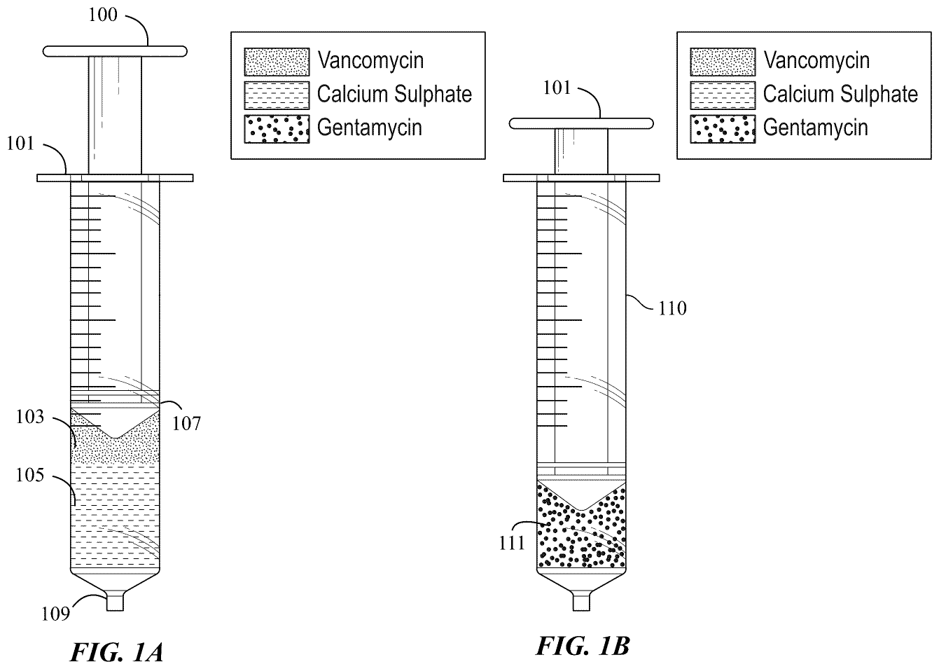

FIG. 1A illustrates a front elevation view of a first syringe 100 of the formatting system 101 for a drug-loaded medical device for preventing surgical infection. The first syringe 100 is loaded with an effective amount of Vancomycin powder 103 and an effective amount of calcium sulfate 105. In some embodiments, the Vancomycin powder 103 is disposed within the first syringe 100 such that it is positioned nearmost the top portion 107 of the first syringe 100, while the calcium sulfate powder is disposed within the first syringe 100 such that it is positioned nearmost the tip 109.

FIG. 1B illustrates a front elevation view of a second syringe 110 of the formatting system 101 for a drug-loaded medical device for preventing surgical infection. The second syringe 110 is loaded with an effective amount of Gentamicin 111. The second syringe 110 is used to extract the effective amount of water which is used during the preparation process of forming the antibiotic beads.

The process of loading the effective amount of Vancomycin powder 103, calcium sulfate 105 and Gentamicin is performed at the time of manufacture, or at least prior to its delivery to the surgeon or other medical staff. In such, the formatting system greatly reduces the time needed to prepare the antibiotic in the operating room.

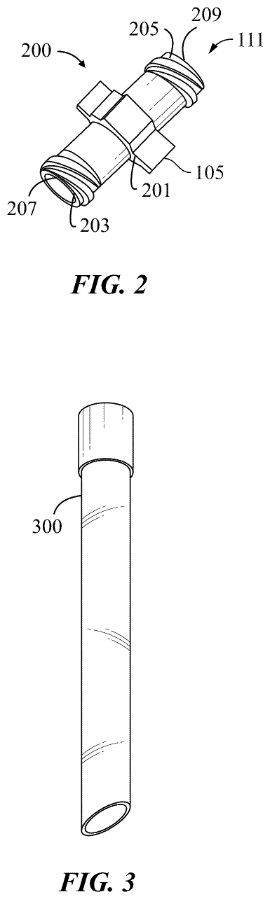

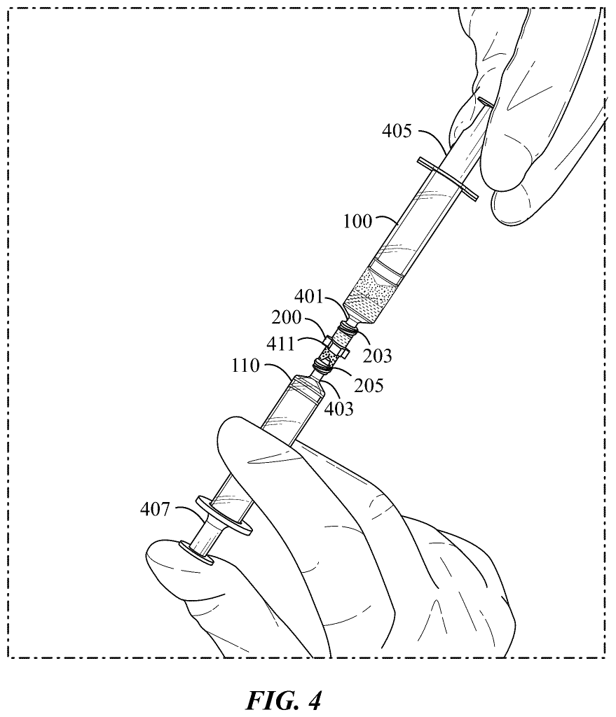

FIG. 2 illustrates the connection device 200 which is used to connect the first syringe and the second syringe. The connection device 200 may be configured as a luer lock connection device 201 which is capable of providing fluid connectivity between the first syringe and the second syringe 110. The connection device 200 may include first and second input ports 203,205 which are positioned at each of a first end 207 and a second end 209. Each of the first and second input ports releasably connect with the tips of the first and second syringes as is illustrated in FIG. 4.

FIG. 3 illustrates a perspective view of the elongated tip 300. The elongated tip 300 enables the precise delivery of the antibiotic agent to the site of infection, or to another precise location on the anatomy of the patient. This includes the ability to access otherwise challenging surgical areas which are not easily accessed using existing methods. The elongated tip 300 enables for the paste to flow in a controllable manner therethrough during the application process, thus reducing waste and ensure the antibiotic is delivered to a precise location.

FIG. 4 illustrates a perspective view of the first syringe 100 and the second syringe 110 connected via the connection device 200 to allow the active ingredients to be mixed as is described by the formatting system for a drug-loaded medical device. As is also described in FIG. 2, the first syringe 100 includes a first tip 401 which connects to a first input port 203 of the connection device 200. The second syringe 100 includes a second tip 403 which connects to the second input port 205. Once connected, the user will mix the active ingredients by operating the plungers 405,407 (i.e., the first plunger 405 and the second plunger 407) of each syringe 100,110. This allows for the back-and-fourth motion of the active ingredients to achieve a mixed antibiotic composition (i.e., the paste). The luer lock mechanism 411 may be operable to lock the connection between the first syringe 100 and second syringe 110.

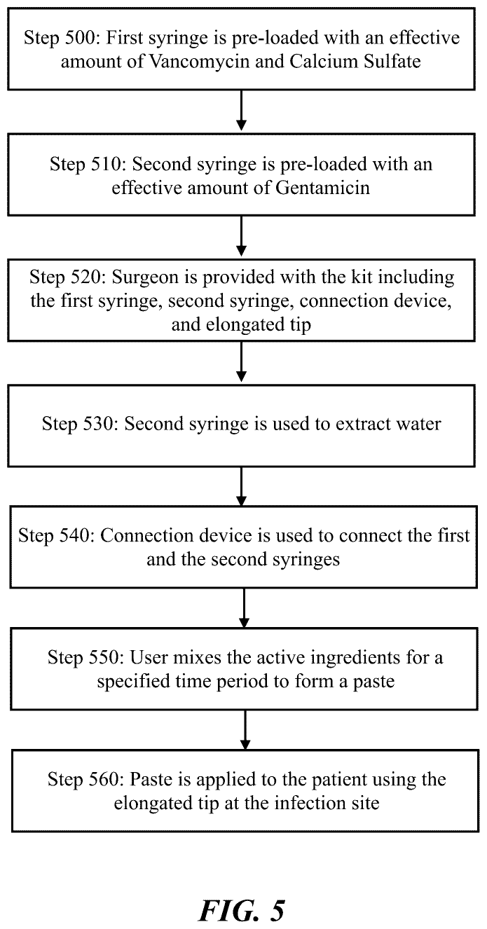

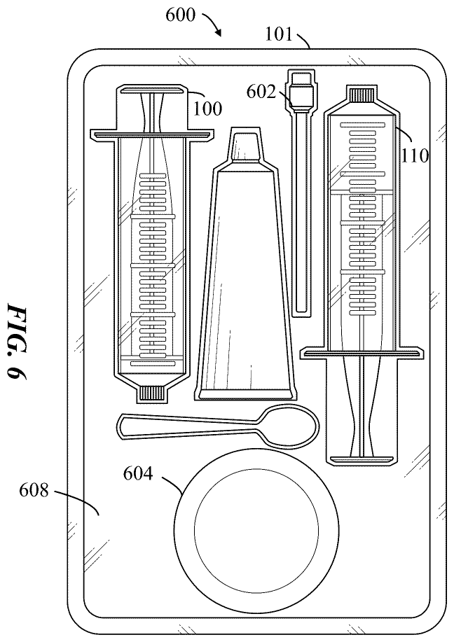

FIG. 5 illustrates a flowchart of a process for preparing and applying the formatting system for a drug-loaded medical device. In step 500, the first syringe is pre-loaded with an effective amount of Vancomycin powder and an effective amount of calcium sulfate. In step 510, the second syringe is pre-loaded with an effective amount of Gentamicin. In step 520, the surgeon is provided with a kit including the first syringe, second syringe, connection device, and elongated tip. In step 530, the second syringe is used to extract a sufficient amount of water into the second syringe. In step 540, the connection device is used to lockingly connect the first syringe and second syringe to the connection device. In step 550, the user mixes the active ingredients for a specified period of time to allow the active ingredients to form a paste. In step 560, the paste is applied to the patient using the elongated tip at the site of infection or other location on the patient's anatomy.

In one example, wherein a 5 cc PTE kit is crated, the device is provided with the first and second syringes 100,110. One component is Vancomycin powder. (5 mg) and a manipulated calcium sulfate (15 ml) this is located in the first syringe 100. The second syringe 110 includes liquid Gentamicin (3 ml). The user will extract 12 ml of sterile water with the second syringe 110. The device and kit comes with a connection luer lock mechanism. The first syringe 100 and the second syringe 110 are locked together and pressed back and forth for 45-seconds mixing the product into a workable paste. One skilled in the arts will readily understand how to modify the above for various additional applications.

The embodiments described in FIGS. 1-5 relate to a pre-loaded formatting system 101 which includes predetermined and preloaded medications. The embodiments described by FIGS. 6-8 enable the medical facility or pharmacy to select medications to be loaded into the formatting system. This is beneficial for providing a customizable formatting system which allows the medical professionals and/or pharmacist to select suitable medications to be loaded into the formatting system. This may be especially useful for patients who have allergic reactions to certain medications or for patients who require specific medication types, amounts, etc.

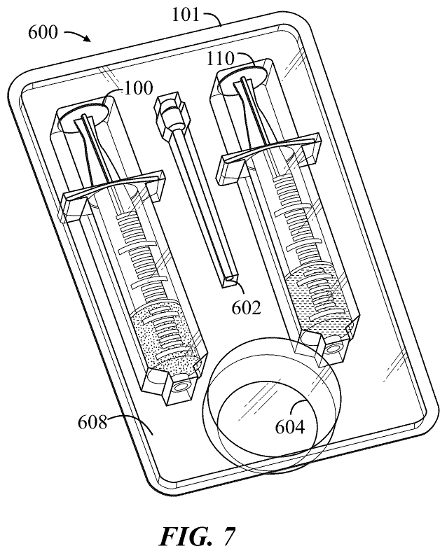

FIG. 6 and FIG. 7 illustrate the kit 600 for the formatting system 101 including the first syringe 100, the second syringe 110, the mixing device 602, and the sterile tray 604. The kit 600 includes each component necessary for the formatting system 101. The kit 600 includes a case 606 which provides a sterile and protective housing to contain the formatting system 101 therein. The first syringe 100 may be loaded with about a sufficient amount of calcium sulfate (such as for example 30 ml of calcium sulfate) while the second syringe 110 is loaded with a sufficient amount of water (such as between 24 ml and 30 ml of sterile water). In some embodiments, a sterile water tube is provided with the kit which may contain between 24 ml and 30 ml of sterile water which is used to mix one or more medications within the sterile tray 604. The mixing device 602 may be used to mix the one or more medications and sterile water within the sterile tray 604.

In some embodiments, the sterile tray 604 may be molded into the interior 608 of the case.

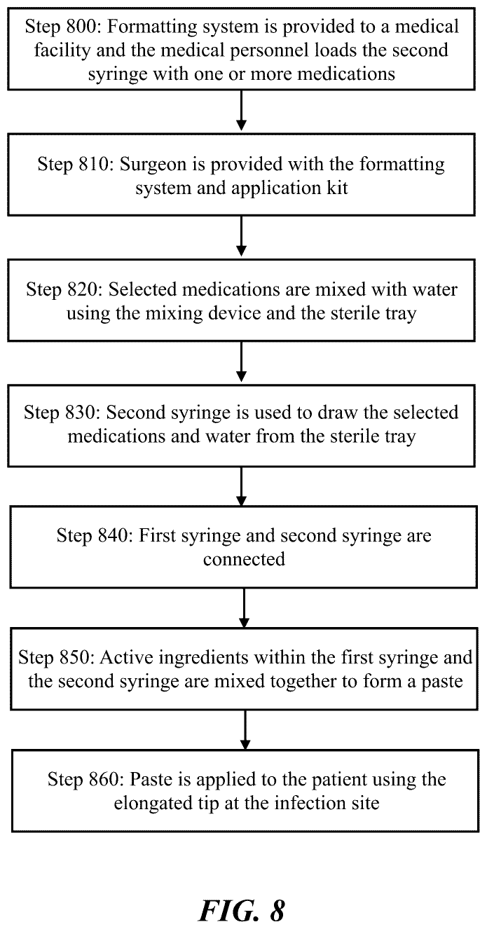

FIG. 8 illustrates a method of utilizing the kit to create the formatting system for use as a means of delivering medications, and specifically antibiotics to a surgical site to prevent infections. In step 800, the formatting system is provided to a medical facility and medical personnel (e.g., a pharmacist) loads the second syringe with one or more medications. In step 810, the surgeon is provided with the kit including the formatting system. In step 820, the selected medications are mixed with a sufficient amount of water using the mixing device and the sterile tray. In step 830, the second syringe used to draw the selected medication(s) and water from the sterile tray. In step 840, the first syringe and the second syringe are connected. In step 850, the active ingredients within the first syringe and the second syringe are mixed together for a specified period of time to allow the active ingredients to form a paste. In step 860, the paste is applied to the patient using the luer lock elongated tip at the site of infection or other surgical site.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A formatting system for a medical device for preventing surgical infection, comprising:
a kit comprising a formatting system, the formatting system including a first syringe including a sufficient amount of calcium sulfate, the formatting system including a second syringe including a sufficient amount of water, the kit including a case to provide a protective and sterile housing;
a sterile tray molded to an interior of the case, the sterile tray to provide a mixing vessel to mix at least one medication with the sufficient amount of water via a mixing device,
wherein the second syringe and the first syringe are connectable to permit the mixing the of the at least one medication, the sufficient amount of water, and the calcium sulfate to form a paste, and wherein an elongated tip facilitates the application of the paste to a patient.

2. The formatting system of claim 1, wherein the connection device is constructed as a luer locking mechanism.

3. The formatting system of claim 2, wherein the luer locking mechanism lockingly engages to the first syringe and the second syringe.

4. The formatting system of claim 1, wherein the sufficient amount of water is between 24 ml and 30 ml.

5. The formatting system of claim 1, wherein the formatting system enables a medical professional to select the one or more medications based on patient parameters.

6. A formatting system for a drug-loaded medical device for preventing surgical infection, comprising:
a first syringe including an effective amount of Vancomycin powder and an effective amount of calcium sulfate;
a second syringe including an effective amount of gentamicin and an effective amount of water, wherein the first syringe and the second syringe are connected via a connection device to permit the mixing of the effective amount of Vancomycin powder, the effective amount of calcium sulfate, the effective amount of water and the effective amount of Gentamicin to form a paste; and
an elongated tip for facilitating the application of the paste to a patient.

7. The formatting system of claim 5, wherein the effective amount of Vancomycin powder for a 5 cc kit is about 0.5 mg.

8. The formatting system of claim 6, wherein the effective amount of calcium sulfate for the 5 cc kit is about 15 ml.

9. The formatting system of claim 7, wherein the effective amount of Gentamicin for the 5 cc kit is about 3 ml.

10. The formatting system of claim 8, wherein the effective amount of water is about 12 ml.

11. The formatting system of claim 5, wherein the connection device is constructed as a luer locking mechanism.

12. The formatting system of claim 10, wherein the elongated tip is effective in applying a bead formed by the paste.

13. A method for providing a formatting system for a medical device for preventing surgical infection, the method comprising the steps of:
providing a kit including the formatting system, the formatting system comprised of a case housing a first syringe, a second syringe, a mixing device, and a sterile tray;
selecting, via a medical professional, at least one medication;
mixing, via the mixing device and sterile tray, the at least one medication with a sufficient amount of water;
loading the sufficient amount of water and the at least one medication into the second syringe;
connecting the first syringe to the second syringe, wherein the first syringe is loaded with a sufficient amount of calcium sulfate;
mixing the contents of the first syringe with the contents of the second syringe to form a paste;
applying, via an elongated tip, the paste to the surgical site to prevent a surgical infection.

14. The method of claim 13, wherein the at least one medication is an effective amount of Vancomycin powder.

15. The method of claim 13, wherein the effective amount of calcium sulfate for the 5 cc kit is about 15 ml.

16. The method of claim 15, wherein the at least one medication is an effective amount of Gentamicin.

17. The method of claim 16, wherein the effective amount of water is about between 12 ml and 30 ml.

18. The method of claim 17, further comprising a luer locking mechanism to connect the first syringe to the second syringe.

19. The method of claim 18, wherein the formatting system is utilized as a bone void filler.

20. The method of claim 19, wherein the elongated tip is effective in applying a bead formed by the paste.
